(19) United States
(12) Patent Application Publication
(10) Pub. No.: US 2012/0045787 A1
(43) Pub. Date: Feb. 23, 2012

(54) FLEXIBLE MICRO-CARRIER SYSTEM

(76) Inventor: Ulrich Boettiger, Boise, ID (US)

(21) Appl. No.: 13/183,379

(22) Filed: Jul. 14, 2011

Related U.S. Application Data
(60) Provisional application No. 61/439,266, filed on Feb. 3, 2011.
(60) Provisional application No. 61/375,227, filed on Aug. 19, 2010.

(51) Int. Cl. C12Q 1/02; C12M 1/34; G01N 21/00

(52) U.S. Cl. 435/29; 436/164; 435/288.7; 422/68.1; 422/400

(57) ABSTRACT

Micro-carrier systems may be used to carry and identify sample materials through an analysis system. Analysis systems may include an image sensor integrated circuit containing image sensor pixels. A channel containing a fluid with particles such as cells may be formed on top of the image sensor. Micro-carriers may be used to carry the cells in the fluid. Micro-carriers may have identifier regions and active regions. Identifier regions may include coded information identifying cells, fluid samples, or other materials carried in the active region. Active regions may carry reagents, trapping agents, cells or other sample materials. Active regions may be formed on a surface of a micro-carrier or may be formed in a cavity inside the micro-carrier. Micro-carriers may include magnetic control structures that can be used to guide, rotate, accelerate or position micro-carriers.

This application claims the benefit of provisional patent application No. 61/439,266, filed Feb. 3, 2011, and provisional patent No. 61/375,227, filed Aug. 19, 2010, which are hereby incorporated by reference herein in their entireties.

### BACKGROUND

This relates generally to analysis systems such as optofluidic microscope systems, and, more particularly, to micro-carriers for carrying specimens through such analysis systems.

Optofluidic microscopes have been developed that can be used to generate images of cells and other biological specimens. The cells are suspended in a fluid. The fluid flows over a set of image sensor pixels in a channel. The image sensor pixels may be associated with an image sensor pixel array that is masked using a metal layer with a pattern of small holes. In a typical arrangement, the holes and corresponding image sensor pixels are arranged in a diagonal line that crosses the channel. As cells flow through the channel, image data from the pixels may be acquired and processed to form high-resolution images of the cells.

In a conventional optofluidic microscope, cells or other samples are identified based on images of the samples themselves. The identification process may require intensive processing or post-processing of image data.

It would be desirable to be able to provide optofluidic microscopes or other analysis systems with systems for simultaneously identifying, carrying and manipulating samples.

### BRIEF DESCRIPTION OF THE DRAWINGS

FIG. 1 is a diagram of an illustrative diagram of micro-carrier systems in an analysis and testing environment in accordance with an embodiment of the present invention.

FIG. 2 is a diagram of an illustrative diagram of micro-carrier systems in a system for imaging and processing cells and other biological specimens in accordance with an embodiment of the present invention.

FIG. 3 is a top view of an illustrative micro-carrier system having multiple functional regions in accordance with an embodiment of the present invention.

FIG. 4 is a top view of an illustrative rectilinear identifier region of a micro-carrier system in accordance with an embodiment of the present invention.

FIG. 5 is a cross-sectional side view of an illustrative portion of a micro-carrier system in the vicinity of an identifier region of the micro-carrier system in accordance with an embodiment of the present invention.

FIG. 6 is a top view of an illustrative round identifier region of a micro-carrier system in accordance with an embodiment of the present invention.

FIG. 7 is a cross-sectional side-view of an illustrative micro-carrier system in the vicinity of an active region of the micro-carrier system in accordance with an embodiment of the present invention.

FIG. 8 is a cross-sectional side-view of an illustrative micro-carrier system in the vicinity of an active region of the micro-carrier system in accordance with an embodiment of the present invention.

FIG. 9 is a cross-sectional side-view of an illustrative micro-carrier system in the vicinity of magnetic control structures in accordance with an embodiment of the present invention.

### DETAILED DESCRIPTION

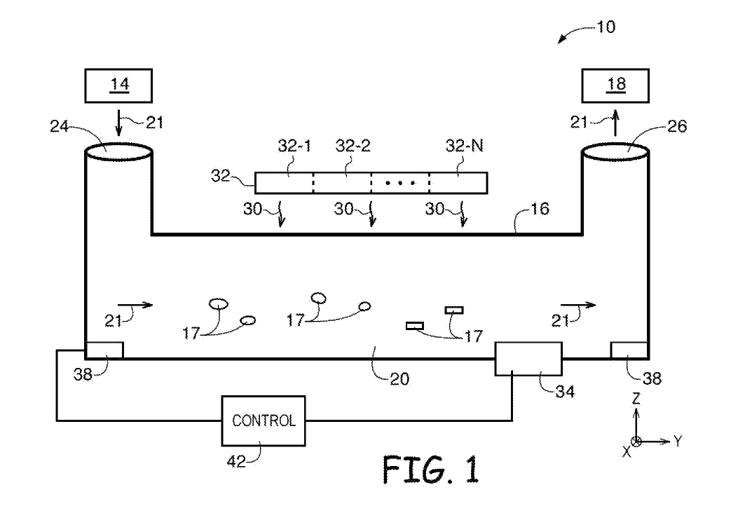

An analysis system of the type that may include cells and other samples such as biological specimens carried on micro-carriers is shown in FIG. 1. As shown in FIG. 1, system 10 may include fluid 20 in fluid channel 16. Fluid channel 16 may be any channel through which fluid 20 may flow (e.g., a blood vessel, digestive tract, plastic, glass or metal tubing as part of a larger system, a channel in an optofluidic microscope, etc.) Channels 16 may have lateral dimensions (dimensions parallel to dimensions x and z in the example of FIG. 1) of a millimeter or less (as an example). The length of each channel (the dimension of channel 16 along dimension y in the example of FIG. 1) may be 1-10 mm, less than 10 mm, more than 10 mm, or other suitable length.

During operation, fluid 20 may flow through channel 16 as illustrated by arrows 21. A fluid source such as source 14 may be used to introduce fluid into channel 16 through entrance port 24. Fluid may, for example, be dispensed from a pipette, from a drop on top of port 24, from a fluid-filled reservoir, from tubing that is coupled to an external pump, from a biological source as pumping from a heart or ingestion through a mouth, etc. Fluid may exit channel 16 through exit port 26 and may, if desired, be collected in reservoir 18. Reservoirs (sometimes referred to as chambers) may also be formed within portions of channel 16.

System 10 may include other components such as image sensor integrated circuit 34, fluid and particle flow control structures 38, external illuminating light sources 32, or other components. Image sensor integrated circuit 34 may be formed from a semiconductor substrate material such as silicon and may contain numerous image sensor pixels. Complementary metal-oxide-semiconductor (CMOS) technology or other image sensor integrated circuit technologies may be used in forming image sensor pixels in integrated circuit 34. Image sensor integrated circuit 34 may include color filters, transparent cover layers or other covering layers. Image sensor integrated circuit 34 may be formed outside of, wholly within, or partially inside and partially outside of channel 16. External illuminating light source 32 may include multiple independent light generating components 32-1, 32-2, 32-3 . . . 32-N for generating light of different colors and frequencies (e.g., laser light, x-rays, etc.) for illuminating micro-carriers 17 inside channel 16.

The rate at which fluid flows through channel 16 may be controlled using fluid flow rate control structures 38. Fluid 20 may contain micro-carriers 17 having a round exterior shape, having an oval exterior shape, having a rectilinear exterior shape or other suitable shape. Micro-carriers 17 may carry materials to be analyzed such as cells, reagents, reactants or other biological elements or particles. Micro-carriers 17 may include portions containing identifying information of the carried materials. Materials carried by micro-carriers 17 may be identified and analyzed while in channel 16 (e.g., as micro-carriers 17 pass by image sensor 34). Control circuitry 42 (which may be implemented as external circuitry or as circuitry that is embedded within image sensor integrated circuit 34) may be used to process the image data that is acquired using integrated circuit image sensor 34.

Micro-carriers 17 may, if desired, be collected using reservoir 18 for later analysis or may be configured to dissolve in fluid 20 after a given amount of time. Collecting micro-carriers in reservoir 18 may include magnetically or electrically capturing micro-carriers 17 in docking stations within channel 16, extracting micro-carriers 17 from exit port 26 with a syringe or other extraction device, collection of bodily fluids such as blood, urine, etc. or other collection methods.

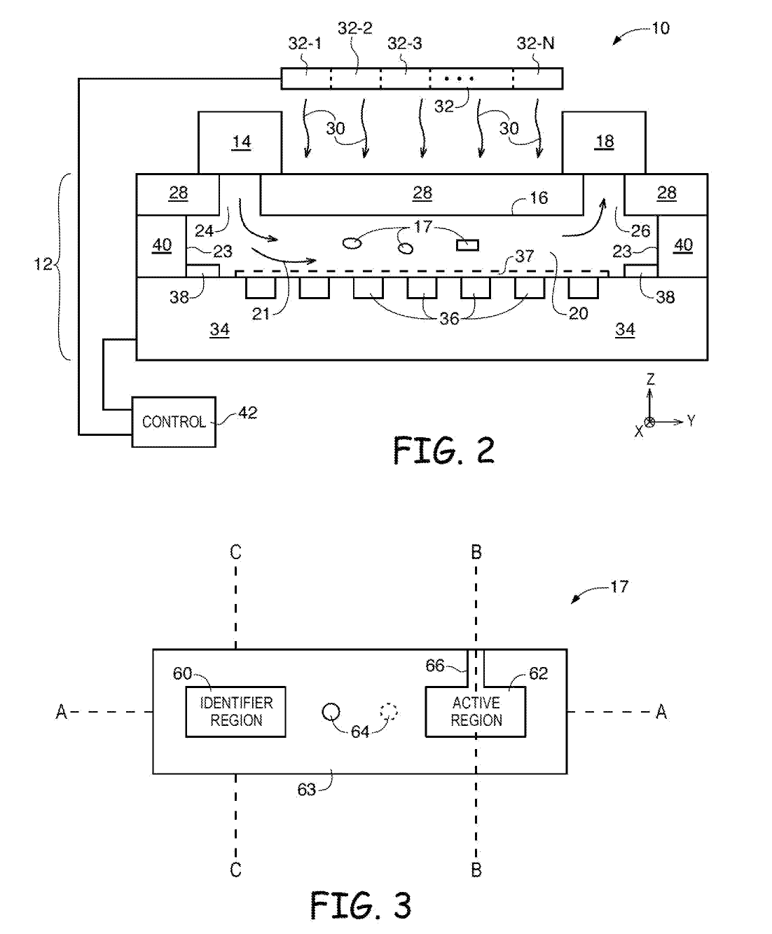

A particular system (discussed as an example herein) of the type that may be used to image and otherwise evaluate cells and other samples such as biological specimens carried on micro-carriers 17 is shown in FIG. 2. As shown in FIG. 2, system 10 may include optofluidic microscope 12. Microscope 12 may include an image sensor integrated circuit such as image sensor integrated circuit 34. Image sensor integrated circuit 34 may be formed from a semiconductor substrate material such as silicon and may contain numerous image sensor pixels 36. Complementary metal-oxide-semiconductor (CMOS) technology or other image sensor integrated circuit technologies may be used in forming image sensor pixels 36 and integrated circuit 34.

Image sensor pixels 36 may form part of an array of image sensor pixels on image sensor integrated circuit 34 (e.g., a rectangular array). Some of the pixels may be actively used for gathering light. Other pixels may be inactive or may be omitted from the array during fabrication. In arrays in which fabricated pixels are to remain inactive, the inactive pixels may be covered with metal or other opaque materials, may be depowered, or may otherwise be inactivated. There may be any suitable number of pixels fabricated in integrated circuit 34 (e.g., tens, hundreds, thousands, millions, etc.). The number of active pixels in integrated circuit 34 may be tens, hundreds, thousands, or more).

Image sensor integrated circuit 34 may be covered with a transparent layer of material such as glass layer 28 or other covering layers. Layer 28 may, if desired, be colored or covered with filter coatings (e.g., coatings of one or more different colors to filter light). Image sensor pixels 36 may be covered with color filter layer 37. Color filter layer 37 may be color filtering material formed individually on image sensor pixels 36 or applied as a flat planar coating covering the lower surface of fluid channel 16. Color filter layer 37 may include portions with red color filters, portions with blue color filters, portions having green color filers, portions having tiled color filters (e.g., tiled Bayer pattern filters, etc.). If desired, color filter layer 37 may include infrared-blocking filters, ultraviolet light blocking filters, visible-light-blocking-and-infrared-passing filters, etc. Structures such as standoffs 40 (e.g., polymer standoffs) may be used to elevate the lower surface of glass layer 28 from the upper surface of image sensor integrated circuit 34. This forms one or more channels such as channels 16. Channels 16 may have lateral dimensions (dimensions parallel to dimensions x and z in the example of FIG. 2) of a millimeter or less (as an example). The length of each channel (the dimension of fluid channel 16 along dimension y in the example of FIG. 2) may be 1-10 mm, less than 10 mm, more than 10 mm, or other suitable length. Standoff structures 40 may be patterned to form sidewalls 23 for channels such as channel 16. Sidewalls 23 and other surfaces of channel 16 may be treated to facilitate or control the flow of fluid 20 through channel 16 (e.g., sidewalls may be plasma treated, coated with hydrophobic material, coated with hydrophilic material, coated with oleophobic material, coated with oleophilic material, etc.).

During operation, fluid 20 flows through channel 16 as illustrated by arrows 21. A fluid source such as source 14 may be used to introduce fluid into channel 16 through entrance port 24. Fluid may, for example, be dispensed from a pipette, from a drop on top of port 24, from a fluid-filled reservoir, from tubing that is coupled to an external pump, etc. Fluid may exit channel 16 through exit port 26 and may, if desired, be collected in reservoir 18. Reservoirs (sometimes referred to as chambers) may also be formed within portions of channel 16.

The rate at which fluid flows through channel 16 may be controlled using fluid flow rate control structures. Examples of fluid flow rate control structures that may be used in system 10 include pumps, electrodes, microelectromechanical systems (MEMS) devices, magnets, etc. If desired, structures such as these (e.g., MEMs structures or patterns of electrodes) may be used to form fluid flow control gates (i.e., structures that selectively block fluid flow or allow fluid to pass and/or that route fluid flow in particular directions). In the example of FIG. 2, channel 16 has been provided with electrodes such as electrodes 38. By controlling the voltage applied across electrodes such as electrodes 38, the flow rate of fluids in channel 16 such as ionic fluids may be controlled by control circuitry 42. Electrodes 38 may also be configured to interact with magnetic control structures attached to micro-carriers 17. Micro-carriers may be guided, directed, held, or otherwise manipulated in three dimensions using magnetic control structures and electrodes 38 through channel 16 or into docks of corresponding shape for mating with micro-carriers 17 within fluid channel 16.

Fluid 20 may contain micro-carriers such as micro-carriers 17. Micro-carriers 17 may include active regions (sometimes referred to herein as sample regions) containing cells or other biological elements, particles or other materials. As micro-carriers such as micro-carriers 17 pass by sensor pixels 36, image data may be acquired. In effect, the micro-carrier may be “scanned” across the pattern of sensor pixels 36 in channel 16 in much the same way that a printed image is scanned in a fax machine. Alternatively, image sensor pixels 36 may be used together to capture static images of micro-carrier 17. As an example, fluid flow rate control structures 38 may be used to hold micro-carrier 17 in a fixed position during capture of light from micro-carrier 17 (e.g., light reflected from active or identifier regions of micro-carrier 17, light emitted by active or identifier regions of micro-carrier 17, etc.). Control circuitry 42 (which may be implemented as external circuitry or as circuitry that is embedded within image sensor integrated circuit 34) may be used to process the image data that is acquired using sensor pixels 36. Because the size of each image sensor pixel 36 is typically small (e.g., on the order of 0.5-5.6 microns or less in width), precise image data may be acquired. This allows high-resolution images of cells such as micro-carriers 17 to be produced. A typical micro-carrier may have dimensions on the order of 10-50 microns (as an example). Portions of micro-carriers 17 carrying cells or other biological material may have dimensions on the order of 0.5-20 microns (as an example).

Portions of micro-carriers 17 may include coded identifying information of the types, quantities, locations, etc. (e.g., using color coded bit patterns on the surface of micro-carriers 17) of materials carried in active regions of micro-carriers 17, identifying information of the processing and analysis history of micro-carriers 17, etc. Coded information may be imaged using image sensor pixels 36 of image sensor integrated circuit 34. Images of coded information on micro-carriers 17 may be used by control circuitry or other external circuitry to identify multiple types of biological samples on a single micro-carrier while micro-carrier is in channel 16 of microscope 12, to identify the analysis and processing history of micro-carriers 17, to identify the spatial orientation of micro-carriers 17, etc. Arrangements in which micro-carriers are imaged are sometimes described herein as an example.

During imaging operations, control circuit 42 (e.g., on-chip and/or off-chip control circuitry) may be used to control the operation of light source 32. Light source 32 may be based on one or more lamps, light-emitting diodes, lasers, or other sources of light. Light source 32 may be a white light source or may contain one or more light-generating elements 32-1, 32-2, 32-3 . . . 32-N that emit different colors of light. For example, light-source 32 may contain multiple light-emitting diodes of different colors or may contain white-light light-emitting diodes or other white light sources that are provided with different respective colored filters. Light source 32 may be configured to emit laser light of a desired frequency or combination of frequencies. If desired, layer 28 and layer 37 may be implemented using colored transparent material in one or more regions that serve as one or more color filters. In response to control signals from control circuitry 42, light source 32 may produce light 30 of a desired color, intensity, polarization or illumination direction. As an example, in response to control signals from control circuitry 42, elements 32-1, 32-2, 32-3 . . . 32-N may be lit sequentially while fluid rate control structures 38 hold micro-carriers 17 in a single position (e.g., so that micro-carriers 17 may be lit from differing angles and in differing colors). Light 30 may pass through glass layer 28 to illuminate the micro-carriers 17 in channel 16. A detailed view of an exemplary micro-carrier such as micro-carriers 17 that may be implemented in test and analysis systems such as system 10 is shown in FIG. 3.

FIG. 3 is a top view of an illustrative micro-carrier system for identifying and transporting chemical, biological or other materials. As shown in FIG. 3, micro-carrier 17 may be formed from carrier structures 63. Carrier structure 63 may have relatively small dimensions. For example, carrier structure 63 may have dimensions of less than 1 mm, less than 1000 microns, less than 250 microns, less than 100 microns, less than 50 microns, etc. Use of relatively small dimensions for carrier structure 63 may allow micro-carrier system 17 to be used in applications where large sizes might become stuck or might otherwise not be acceptable. For example, the small dimensions of carrier structure 63 may allow carrier structure 63 to be deployed in the blood stream of a patient.

Carrier structure 63 of carrier 17 may include functional regions such as identifier region 60 and active region 62. Identifier region 60 may include coded information (e.g., identifying information in the form of color filters or other light absorbing, reflecting or polarizing structures formed on the surface of micro-carrier 17). Identifier region 60 may be formed in one region of micro-carrier 17 or may have portions in multiple regions of micro-carrier 17 (e.g., two or more identifier regions spatially separated on carrier structure 63 to aid in determining the spatial orientation of micro-carrier 17). Coded information in identifier region 60 may be used to identify materials carried in active region 62, may be used to record the history of micro-carrier 17 (e.g., previous tests, identifying information of a patient from which material carried in active region 62 was taken, etc.) may be used to identify sub-regions of active region 62 carrying different samples, may be used to determine the spatial orientation of micro-carrier 17, or may encode other information.

Active regions such as active region 62 of sample carrier 17 of FIG. 3 (sometimes referred to herein as sample gathering regions, sample chambers, fluid sample chambers, sampling regions, reactant regions, sample analysis regions, etc.) may be used to gather samples through openings such as channel 66. For example, if carrier 17 is placed or immersed in a fluid, a sample of the fluid may be gathered by sample gathering region 62 through channel 66. Reactants (e.g., one or more reactant coatings) may be provided in region 62 to react with fluid samples and therefore assist in the analysis of the fluid samples.

Micro-carrier 17 may include one or more magnetic control structures 64 (e.g., magnets that may be used as “handles” for magnetically three-dimensionally positioning, orienting and directing micro-carriers 17). Active region 62 may be formed on an exterior surface of micro-carrier 17 or may be formed as a cavity (sometimes referred to herein as a chamber or sample gathering chamber) inside micro-carrier 17. Active regions 62 of that are formed as cavities inside micro-carrier 17 may have an associated access ports such as access port 66 of FIG. 3. Access port 66 may have properties designed to allow desired materials to enter active region 62 (e.g., the size, shape, surface features, surface coatings, etc of access port 66 may inhibit or encourage entrance of certain fluids or other materials). Identifier region 60, active region 62 and magnetic control structures 64 of micro-carrier 17 may facilitate simultaneous, real time, in-situ identification, controlled manipulation and targeted, controlled analysis of multiple samples carried in active region 62 in analysis and test systems such as optofluidic microscope 12 of FIG. 2. Micro-carriers 17 may be made small enough for ingestion (and later collection and analysis), small enough for injection into bodily tissues or blood vessels, or for insertion into other environments for later extraction and analysis, as described in connection with system 10 of FIG. 1.

Micro-carrier 17 may be formed from glass, silicon, plastic, or other suitable materials or combinations of materials. The materials that are used in forming the carrier structure for micro-carrier 17 may be transparent to facilitate imaging of fluid samples that are captured within micro-carrier 17. Bio-compatible materials may be used in forming the carrier structure for micro-carrier 17 to allow micro-carrier 17 to be introduced into blood vessels or other biologically sensitive environments. If desired, the carrier structure may be formed from materials that are suitable for patterning using mass production techniques such as semiconductor fabrication techniques, advanced printing techniques (e.g., ink-jetting) or other patterning techniques.

Micro-carrier 17 may be formed a single structure or may be a formed by attaching two or more layers allowing the formation of cavities between portions of the layers. In one preferred embodiment, micro-carrier 17 may preferably be formed using wafer based silicon processing techniques and advanced packaging technology. Many micro-carriers 17 may be formed on a single silicon wafer and singulated into individual carriers using wafer thinning, lithography, dry or wet etching. Forming micro-carriers 17 from a single silicon wafer may help avoid the need for mechanical dicing steps during formation of micro-carriers. During formation of micro-carriers 17, an intermediate carrier (e.g., a film or other wafer) may be used. Micro-carrier 17 may be made to be wholly or partially transparent by including light absorbing or color filtering layers on the surface of micro-carrier 17. As an alternative to permanent material such as glass or silicon, micro-carrier 17 may be formed from a cellulose material or other fluid-soluble material designed to dissolve in a fluid (e.g., material that is configured to dissolve after a certain amount of time inside a patient's digestive track if not extracted for analysis).

Micro-carriers 17 may have dimensions on the order of 10-50 microns (as an example) with a thickness on the order of 10 microns or less (as an example). Other dimensions may be used for forming carrier structure that makes up micro-carriers 17 if desired. For example, a micro-carrier 17 may be formed from a carrier structure with a maximum dimension that is less than 1000 microns, less than 500 microns, less than 100 microns, in the range of 10-100 microns, etc. Multiple micro-carriers 17 each having different size and exterior shape may be used in a single analysis and test system such as system 19. Micro-carriers 17 may, for example, have a substantially rectilinear shape (as in the example of FIG. 3), may have a substantially rounds exterior shape, may have a substantially oval exterior shape or may have any other suitable exterior shape. The overall exterior shape of micro-carriers such as micro-carrier 17 may be used to convey information (i.e., certain shapes may carry corresponding types of materials), or may be used for mechanical orientation or selective docking of micro-carriers 17 (i.e. in docks of corresponding shape inside a channel such as channel 16 of FIG. 1).

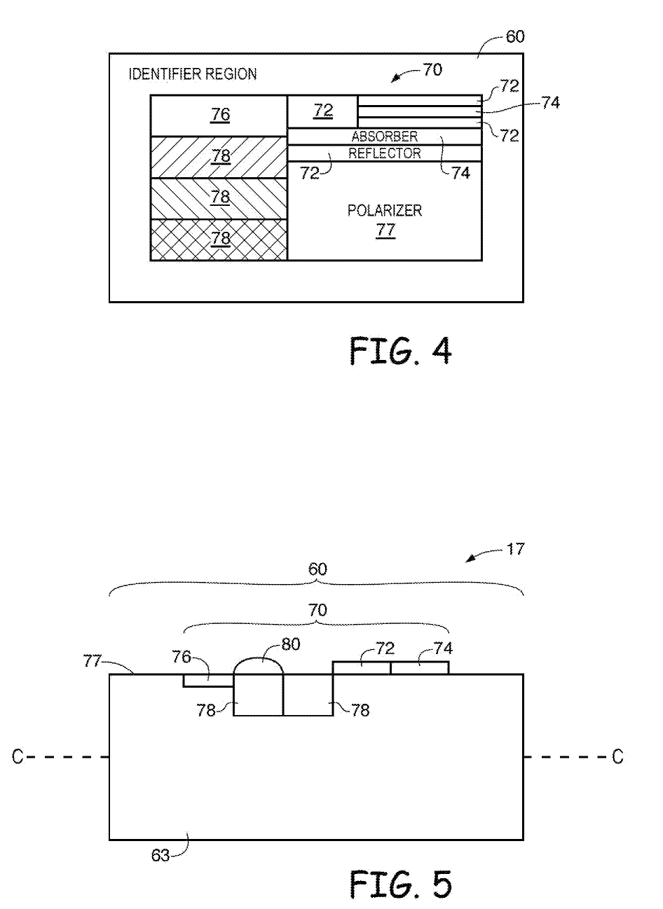

FIG. 4 is a top view of an identifier region such as identifier region 60 of FIG. 3. As shown in FIG. 4, identifier region may include information coding structures 70 (sometimes referred to herein as coded information 70). Information coding structures 70 may be formed from materials selected from the group consisting of: color coded materials, patterned opaque structures, optical filters, polarizers, color filter array structures, structures covered with microlenses, and photonic nano-structures. Coded information 70 in identifier region 60 may represent simple binary coding (i.e., black and white materials such as absorbing and reflecting materials 74 and 72 respectively), may use color coding or may use polarization coding (i.e., coding information by forming portions of identifier region 60 covered with different light polarizing materials). Information coding structures 70 may include regions such as reflective regions 72 and light absorbing regions 74. Reflective regions 72 and absorbing regions 74 may be configured to encode information more complex than a simple binary string (e.g., absorbing and reflecting portions may have differing sizes as in a bar code). Reflective regions 72 may be formed on the surface of micro-carrier 17 using metal film patterning, photo-lithography and etching, screen printing or ink-jetting of reflective material or other methods. Absorbing regions 74 may be formed by screen printing light absorbing material (or depositing material using other deposition methods such as ink-jetting) onto the surface of micro-carrier 17. Identifier region 60 may include substantially transparent portions such as transparent portion 76, portions coated with light polarizing materials such as polarizing portion 77 and portions configured to absorb light of different color such as color filter regions 78.

As shown in the cross-sectional side view of FIG. 5 (taken along line C of carrier structure 63 of micro-carrier 17 of FIG. 3) information coding structures 70 of identifier region 60 may optionally include microlenses 80 covering color filter regions 78. Information coding structures 70 may be formed from materials selected from the group consisting of: color coded materials, patterned opaque structures, optical filters, polarizers, color filter array structures, and structures covered with microlenses. Color filter regions 78 and microlenses 80 may be formed as separate structures added to the surface of micro-carrier 17 or may be formed as an integrated portion of micro-carrier 17. In one preferred embodiment, color filter regions 78, polarizing portions 76, absorbing regions 74, reflecting regions 71, microlenses 80 and transparent portions 77 may be formed during a wafer level processing that includes formation of active region 72 of micro-carrier 17 (i.e., all portions of micro-carrier 17 may be formed as integrated portions of a single silicon die). Color filter regions 78 of FIGS. 4 and 5 may be patterned to form red-green-blue (RBG), cyan-magenta-yellow-key (CMYK), infrared or other filter patterns. In the example of FIG. 5, color filter regions 78 may be formed with or without microlenses 80. Identifier region 60 may include any combination of color filter regions 78, polarizing portions 76, absorbing regions 74, reflecting regions 72, microlenses 80 and transparent portions 77.

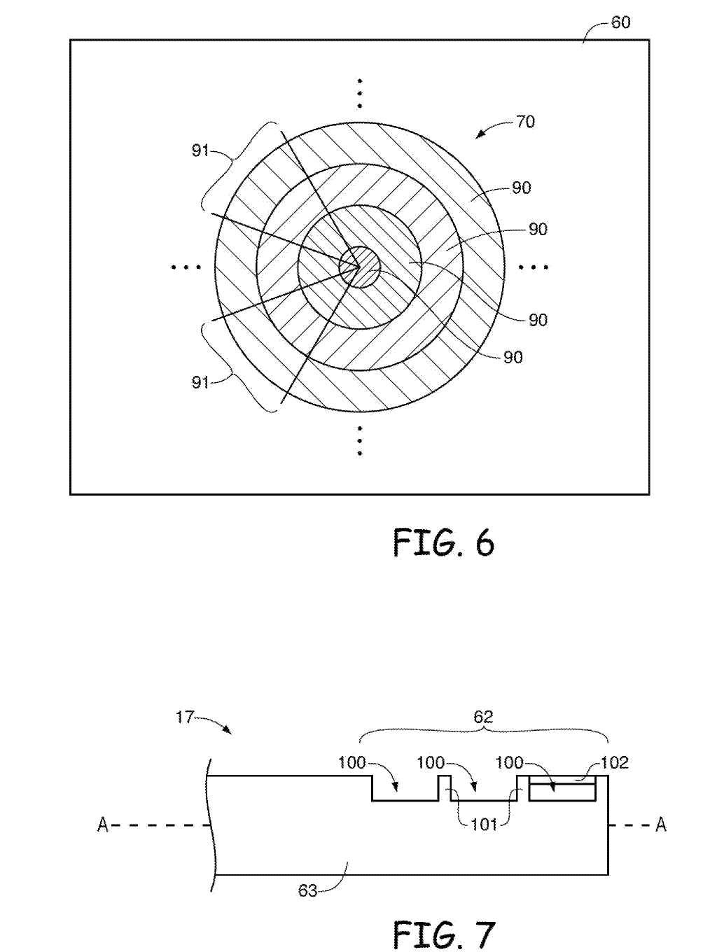

The size, complexity, orientation, shape, etc. of identifier region 60 may be optimized differently for different applications (e.g., using micro-carriers in different fluids, in different analysis systems, for carrying different biological materials, etc.). Identifier region 60 containing coded information 70 may (as shown in FIG. 5) be substantially rectilinear in shape or may substantially round in shape as shown in FIG. 6. Information coding structures 70 of identifier region 60 of FIG. 6 may include one or more circular regions 90. Circular regions 90 may include color filter regions, metal light blocking regions, regions coated with light absorbing material, transparent regions, light polarizing regions or other regions. Coded information 70 of identifier region 60 may be arbitrarily complex and may correspond to the complexity of materials carried in active region 62. For example, coded information 70 in identifier region 60 may be used to identify materials carried in active region 62, may be used to record the history of micro-carrier 17 (e.g., previous tests, identifying information of a patient from which material carried in active region 62 was taken, etc.) may be used to identify sub-regions of active region 62 carrying different samples, or may encode other information. Circular regions 90 may include one or more sub-regions such as sub-regions 91 that encode different information from other sub-regions 91. In the example of FIG. 6, identifier region 60 having four circular code regions is merely exemplary and identifier regions 60 may have more or less than four circular regions and regions having other shapes.

FIG. 7 is a cross-sectional side view of a portion of carrier structure 63 of micro-carrier 17 of FIG. 3 (along line A) in the vicinity of active region 62. As shown in FIG. 7, active region 62 may have multiple portions 100 (e.g., regions that are separated from each other with walls or other separating structures 101). Active region 62 may have any number of portions 100 (e.g., two portions, two or more portions, three portions, three or more portions, four portions, four or more portions, ten or more portions, twenty or more portions, fifty or more portions, 10-100 portions, 96 portions, hundreds of portions, more than 100 portions, etc.) Portions 100 of active region 62 may be used to attach different materials (e.g., reagents, reactants, cells, pharmaceuticals, viruses, bacteria, antigens or other materials). Portions 100 of active region 62 may be used to attach different concentrations of a single material. As an example, portions 100 may be used as a microscopic version of macroscopic pharmaceutical trays in which multiple drugs are exposed to a virus, cancer cells, etc. to test drug viability.

Micro-carrier 17 having a microscopic tray of pharmaceuticals that may be submerged in fluid 20 of FIG. 2 may facilitate rapid parallel testing of multiple drugs. Some portions 100 of active area 62 may (as shown in FIG. 7) have a time-released coating such as time-released coating 102. Time-released coating 102 may be formed from a cellulose material or other fluid-soluble material designed to dissolve over time while submerged in a fluid such as fluid 20 of FIGS. 1 and 2 thereby exposing some portions of active region 62 to fluid 20 at a time later than the time of immersion of micro-carrier 17 in fluid 20. Multiple time-released coatings such as time-released coating 102 may dissolve at different rates thereby exposing different portions 100 to fluid 20 at different times. In the example of FIG. 7, active region 62 is formed on the surface of micro-carrier 17. Time released coating 102 may, if desired, be formed from heat activated material, light activated material or other material designed to be activated or deactivated in an analysis environment for exposing some portions of active region 62 to fluid 20 at a time later than the time of immersion of micro-carrier 17 in fluid 20. An active region may be formed on the surface of micro-carrier 17 by screen printing, etching, ink-jetting, or other suitable methods. Identifier regions such as identifier region 60 of FIG. 3 may be have coded information 70 corresponding to the quantities, concentrations, deposition history, etc. of materials in portions 100 of active region 62.

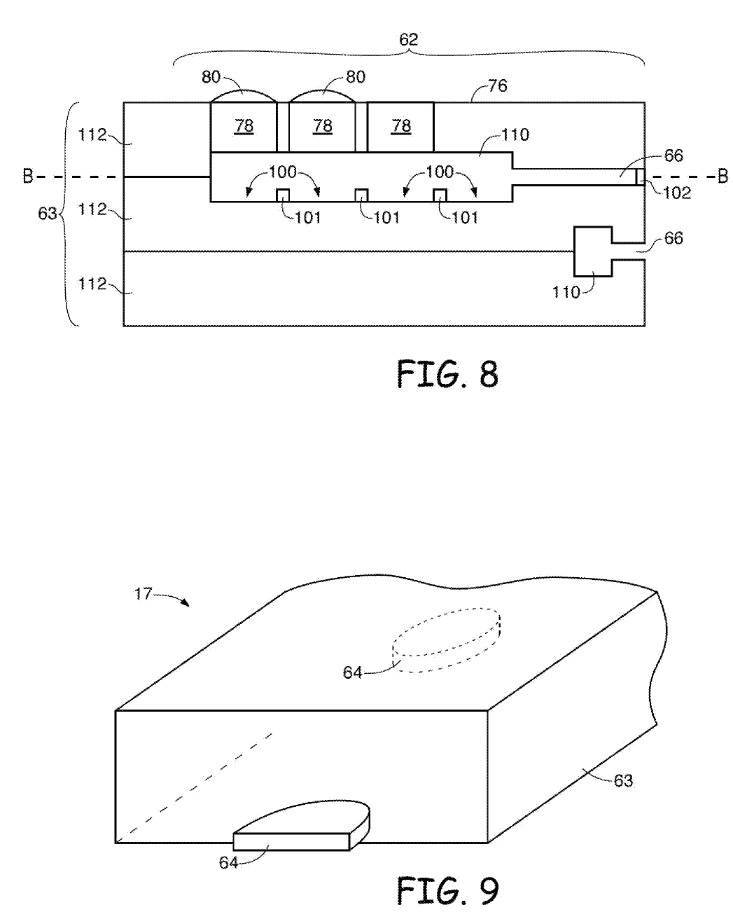

As described in connection with FIG. 3, carrier structure 63 of micro-carrier 17 may be formed from multiple layers such as layers 112 of FIG. 8. As shown in FIG. 8, (a cross-sectional side view of micro-carrier 17 of FIG. 2 taken along line B of FIG. 2) active region 62 may, if desired, be formed using one or more cavities such as cavities 110 (sometimes referred to herein as a chambers, sample gathering chambers, or fluid sample chambers). Cavities 110 may have corresponding access ports such as access port 66. Access ports 66 may have a time-released coating such as time-released coating 102 that prevents fluid 20 from entering sample gathering region 110 before time-released coating 102 dissolves in fluid 20. Time-released coating 102 may be formed from a cellulose material or other fluid-soluble material designed to dissolve over time while submerged in a fluid such as fluid 20 of FIGS. 1 and 2 thereby exposing some portions of active region 62 to fluid 20 at a time later than the time of immersion of micro-carrier 17 in fluid 20. Alternatively, time released coating 102 may, if desired, be formed from heat activated material, light activated material or other material designed to be activated or deactivated in an analysis environment for exposing some portions of active region 62 to fluid 20 at a time later than the time of immersion of micro-carrier 17 in fluid 20 or for exposing some portions of active region 62 to fluid 20 for a shorter duration than other portions of active region 62. Layers 112 of micro-carrier 17 may be assembled using wafer level processing techniques, ink-jetting, row-to-row printing or other processing techniques to enable the creation of cavities (reservoirs) 110. Cavities 110 may be used to pre-deposit materials (e.g., reagents, cell samples, etc.) prior to immersion of micro-carrier 17 in a fluid such as fluid 20 of FIGS. 1 and 2. Targeted reactions of particles or cells in active region 62 with fluid 20 gathered by fluid sample chambers 110 following immersion of micro-carrier 17 in fluid 20 may be observed real-time as in the case of an optofluidic microscope (see FIG. 2) or may be later observed following collection of micro-carriers 17 (as in the case of ingestion of micro-carriers 17). Identifier regions such as identifier region 60 of FIG. 3 may include coded information 70 corresponding to the types, quantities, concentrations, deposition history, etc. of materials pre-deposited in cavities 110 of active region 62.

Active regions 62 may have portions 100 on the surface of micro-carrier 17 (see FIG. 7) or may have portions 100 internal to cavities 110 of micro-carrier 17 as shown in FIG. 8. Portions 100 of fluid sample chambers 110 may have corresponding optical filters such as color filter regions 78, microlenses 80 or transparent regions 76 to facilitate imaging of materials by filtering, focusing or otherwise manipulating light entering chambers 110 of active region 62 using (for example) image pixels 36 of image sensor integrated circuit 34 of FIG. 2.

FIG. 9 is a cross-sectional side view of a portion of carrier structure 63 of micro-carrier 17 of FIG. 3 in the vicinity of magnetic control structures 64. As shown in FIG. 9, magnetic control structures may be partially embedded in a surface of carrier structure 63 or may be formed entirely within carrier structure 63. Multiple magnetic control structures may be formed in several positions within carrier structure 63 (e.g., one magnet each near opposing top and bottom surfaces of carrier structure 63, one magnet each near opposing side walls of micro-carrier 17, etc.). Magnetic control structures 64 may be implanted in carrier structure 63 or may be formed as an integral portion of carrier structure 63. Magnetic control structures 64 may interact with magnetic fields generated using, for example, electrodes 38 of FIGS. 1 and 2. Magnetic control structures 64 may be used to three-dimensionally move, guide, direct, hold, or otherwise manipulate micro-carriers such as micro-carrier 17 within a fluid such as fluid 20 of FIGS. 1 and 2 (e.g. to move micro-carrier 17 through channel 16, to direct micro-carriers to docking stations within channel 16, to stir or otherwise rotate micro-carriers within fluid 20, to move micro-carrier 17 into a position of best focus with respect to image sensors for capturing light from micro-carrier 17, etc.).

Various embodiments have been described illustrating a micro-carrier system for use in carrying and identifying materials to be analyzed (e.g., imaged, exposed to other materials, etc.) through an analysis system such as an optofluidic microscope. The micro-carrier system may include an active region for carrying the material to be analyzed and an identifier region having coded information for identifying the material itself (e.g., types, compositions, quantities, concentrations, deposition history, etc. of materials) or for identifying previous analyses performed on the materials, etc. The active region of the micro-carrier system may be formed on a surface of the micro-carrier system or in a cavity of chamber within the micro-carrier system. The active region may have multiple separate portions for carrying different materials, material samples from different sources, materials in different concentrations, etc. The active region (sample gathering region) may have optically functional elements such as microlenses, color filters, polarizers, etc. that control light entering the active region. The micro-carrier system may be formed from a single structure such as glass or a single silicon die. The micro-carrier system may have an overall exterior shape configured to match the shape of a docking station in the analysis system.

The coded information in the identifier region of the micro-carrier system may be formed using information coding structures such as color filter elements, microlenses or other light absorbing or light reflecting materials formed on the carrier (e.g., on the silicon die) and may be readable using an image sensor in an optofluidic microscope or other imaging device. Analysis systems (e.g., optofluidic microscopes) may gather light that comes from the material in the active region. Gathering light that comes from the material in the active region may include gather images of materials in active regions of micro-carriers during analysis, gathering light emitted by materials in active regions, etc. For example, an image of a fluid sample (gathered by a fluid sample chamber in the carrier structure of a micro-carrier when the micro-carrier is immersed in a fluid) may be obtained using an optofluidic microscope. Images of active regions gathered by analysis systems may also include images of a reactant that has reacted with a fluid sample in a fluid sample chamber of a micro-carrier.

The micro-carrier system may have carrier structures (substrates) with maximum lateral dimensions of less than 1000 microns, less than 500 microns, less than 100 microns, less than 50 microns, in the range of 1-100 microns, in the range of 10-50 microns, more than 100 microns or any other suitable size. Micro-carrier systems may include magnetic control structure for use in guiding or three-dimensionally positioning the micro-carrier system within an analysis system or holding the micro-carrier in a docking station in fluid channels in the analysis system.

The foregoing is merely illustrative of the principles of this invention which can be practiced in other embodiments.

What is claimed is:

1. A micro-carrier system for gathering fluid samples for analysis with an analysis system, comprising:
a carrier structure;
a sample gathering region on the carrier structure that is configured to gather fluid samples when the micro-carrier system is immersed in a fluid; and
an identifier region on the carrier structure having coded information, wherein the identifier region is configured to identify the fluid samples.

2. The micro-carrier system defined in claim 1 wherein the analysis system comprises an optofluidic microscope and wherein the carrier structure is configured to pass through fluid channels in the optofluidic microscope.

3. The micro-carrier system defined in claim 2 wherein the micro-carrier structure is configured to have an overall exterior shape that matches a corresponding shape of a docking station in the fluid channels.

4. The micro-carrier system defined in claim 1 wherein the carrier structure has a maximum dimension of less than 100 microns.

5. The micro-carrier system defined in claim 1 further comprising reactant in the sample gathering region.

6. The micro-carrier system defined in claim 1 wherein the carrier structure is formed from a material selected from the group consisting of: transparent material, silicon, glass, and fluid-soluble material.

7. The micro-carrier system defined in claim 1 further comprising a fluid-soluble material that prevents the fluid samples from entering the sample gathering region before the fluid-soluble material dissolves in the fluid.

8. The micro-carrier structure defined in claim 1 wherein the identifier region comprises a plurality of portions and wherein the plurality of portions are configured to identify the orientation of the micro-carrier structure.

9. The micro-carrier system defined in claim 1 further comprising at least one magnetic control structure for manipulating the micro-carrier system.

10. The micro-carrier system defined in claim 1 wherein the identifier region includes at least one color filter on the carrier structure.

11. The micro-carrier system defined in claim 1 wherein the identifier region includes at least one microlens on the carrier structure.

12. The micro-carrier system defined in claim 1 wherein the sample gathering region comprises a chamber in the carrier structure, the micro-carrier system further comprising at least one optically functional element that controls light entering the chamber.

13. A micro-carrier system for analyzing a material, comprising:
a carrier structure;
a sample region on the carrier structure that is configured carry the material; and
an identifier region on the carrier structure having information coding structures that identify the material, wherein the carrier structure has a maximum dimension of less than 1000 microns.

14. The micro-carrier system defined in claim 13 wherein the information coding structures include information coding structures selected from the group consisting of: color coded materials, patterned opaque structures, optical filters, polarizers, color filter array structures, structures covered with microlenses, and photonic nano-structures.

15. The micro-carrier system defined in claim 13 wherein the carrier structure is formed from a material selected from the group consisting of: transparent material, silicon, glass, and fluid-soluble material.

16. The micro-carrier system defined in claim 13 further comprising a reactant and a fluid-soluble material that prevents the material from contacting the reactant before the fluid-soluble material dissolves in a fluid and that is configured to dissolve after exposure to the fluid for a period of time to allow the material to contact the reactant.

17. A method for analyzing a material using a micro-carrier system having an active region and an identifier region on a carrier structure, wherein the active region is configured to carry the material to be analyzed, the method comprising:
with an analysis system, gathering light that comes from the material in the active region; and
gathering an image of the identifier region.

18. The method defined in claim 17 wherein the active region comprises a fluid sample chamber that gathers a fluid sample when the carrier structure is immersed in a fluid and wherein gathering light that comes from the material comprises gathering an image of the fluid sample.

19. The method defined in claim 17 wherein the active region comprises a reactant that has reacted with a fluid sample and wherein gathering light that comes from the material comprises gathering an image of the reactant.

20. The method defined in claim 17 wherein gathering an image of the identifier region comprises imaging coded information with the analysis system.
